(19) United States
(12) Patent Application Publication
(10) Pub. No.: US 2014/0368822 A1
(43) Pub. Date: Dec. 18, 2014

(54) MEDIA ABSORBENCY DETERMINATION

(71) Applicant: BURBIDGE PTY LTD, West End, Queensland (AU)

(72) Inventor: Allan D. Morrison, Gumdale (AU)

(21) Appl. No.: 14/368,219

(22) PCT Filed: Dec. 26, 2012
(86) PCT No.: PCT/AU2012/001603

§ 371 Date: Jun. 23, 2014

Related U.S. Application Data
(60) Provisional application No. 61/582,092, filed on Dec. 30, 2011.

(51) Int. Cl. G01N 21/59; G01N 33/49

(52) U.S. Cl. CPC G01N 21/59; G01N 33/49
USPC 356/432

(57) ABSTRACT

A method for estimating a quantity of sample material borne by an absorptive substrate. A first radiation is directed onto the substrate. The radiation interacts with the substrate to an extent relative to anisotropy of the substrate. Interaction of the radiation with the substrate is measured at a plurality of measurement sites of the substrate to obtain a first measurement. Then a sample is applied to the substrate to cause absorption of sample material into the substrate at at least one of the measurement sites. After absorption of the sample by the substrate, a second radiation is directed onto the substrate. Interaction of the radiation with the substrate and sample is measured, to obtain a second measurement. The first measurement is compared with the second measurement to estimate a quantity of sample material borne by the substrate.

### CROSS-REFERENCE TO RELATED APPLICATIONS

This application claims the benefit of U.S. Provisional Patent Application No. 61/582,092 filed 30 Dec. 2011, which is incorporated herein by reference.

### TECHNICAL FIELD

The present invention relates to assessing the amount of a sample material borne in an absorbent substrate, by comparing (a) a screening assessment of the substrate before the sample is applied, to (b) an assessment of the substrate after the sample is applied. In particular, the invention relates to dried liquid biological samples collected on absorbent paper or similar media.

### BACKGROUND OF THE INVENTION

There exists a range of processes for handling dried biological samples on absorbent substrates such as filter paper sheets or cards. Such samples can include blood, buccal samples or urine. Uses of such samples include newborn screening, forensic data collection, drug metabolism investigation, pathology assay tests, and the like. There are a wide variety of new applications emerging that benefit from this use of dried samples.

Instrumentation used in initial laboratory handling of dried samples on filter paper cards typically involves a punch device that punches small pieces from the dried sample held on the filter paper, typically in the shape of disks, into receiving wells of laboratory plates for processing. The instrumentation associated with the punch typically creates a database of information confirming the identification of the sample from which the disk is punched, the location of these punched disks in the respective wells of the receiving plates, and the identity of the receiving plates.

Assay methodologies have been developed using these punched disks to generate valuable information regarding the status of the source of the samples. Dried samples on media and particularly dried blood on filter paper have also been used in qualitative assays, where the objective is to identify the presence of a component substance in the sample.

The drying of the sample on the cards after collection is usually achieved by air-drying, often overnight, but in any event for a minimum of 2 hours under standard ambient laboratory conditions of temperature and humidity.

In the case of blood, when a disk of a fixed diameter is punched from a dried blood spot on filter paper, it has in the past been speculated that there are a number of factors that could determine the amount of blood material trapped within that disk. These have included the volume of blood actually spotted, the hematocrit level (percentage of red blood cells) of the blood, the environment within which the blood is allowed to dry, the time taken to dry, the anisotropic distribution of blood throughout the blood spot, and the site within the blood spot from which the disk is punched, amongst other factors.

While the use of dried blood samples on filter paper has been the basis for neonatal screening since the 1960s, the associated assays have in the past been of a less precise nature, and the need for levels of accuracy better than +/−15% has not been a high priority. However, the use of dried blood spots on filter paper is increasingly spreading into other applications, such as quantitative assay methods including drug discovery. In many such applications there is an increasing need for accuracy in determining the volume or concentration of an analyte of interest. Improving the accuracy and lowering the level of uncertainty associated with such applications, including assay and newborn screening outcomes, will lead to reduced laboratory costs and improved health benefits.

This issue associated with the need for a more accurate understanding of the amount of dried sample material at a specific location on a filter paper card applies not only to downstream processes that involve punching, but also to those that involve other approaches, such as those referred to as direct absorption, whereby liquid is forced under pressure through a specific location on a biological sample on a filter paper card.

In recent times, as a result of the pressures from the bioanalysis sector in particular, various studies have focussed on identifying the extent of the relationship between the factors identified above, and the amount of blood material that can be found in blood spots.

The vast majority of laboratories that use dried biological samples on filter paper use a punching approach to remove a fixed-size standard disk from the biological spot, for use in their assay or other laboratory process. A typical spot diameter will range from 7 mm-13 mm. The typical punch sizes range from 1.2 mm-6 mm. These laboratories use manual methods to decide upon a site within the biological spot to punch.

A method to measure the density of dried blood material in filter paper is the subject of U.S. Pat. No. 8,273,579, the content of which is incorporated herein by reference. That technology provides for light to be transmitted through the dried sample on filter paper, and for the amount of light so transmitted to be measured on the other side of the filter paper. That scanning technology works on the principle that the higher the level of dried blood material trapped in the filter paper, the lower the level of transmitted light. The measurement of the transmitted light in a particular area on the filter paper card could then be compared in each laboratory with a threshold level established by that laboratory as its acceptable maximum level. Given vagaries in the measurement, laboratories usually establish a suitable margin for error.

A number of recent scientific papers have reported differences in absorption capacities and rates between the different paper types now commercially available, between batches in each type, and between sections of paper within the same batch. These manufacturer-to-manufacturer variations, and batch-to-batch variations within the same manufacturer, are typically at least +/−4-5%.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY OF THE INVENTION

According to a first aspect the present invention provides a method for estimating a quantity of sample material borne by an absorptive substrate, the method comprising:

prior to absorption of a sample by the substrate, directing a first radiation onto the substrate, the first radiation being configured to interact with the substrate to an extent relative to anisotropy of the substrate, and measuring interaction of the first radiation with the substrate at a plurality of measurement sites of the substrate to obtain a first radiation interaction measurement;

applying a sample to the substrate to cause absorption of sample material into the substrate at at least one of the measurement sites;

after absorption of the sample by the substrate, directing a second radiation onto the substrate, the second radiation being configured to interact with the substrate and to interact with the absorbed sample borne by the substrate to an extent relative to anisotropy of the substrate and the absorbed sample, and measuring interaction of the radiation with the substrate and sample at the plurality of measurement sites of the substrate to obtain a second radiation interaction measurement; and

comparing the first radiation interaction measurement with the second radiation interaction measurement to estimate a quantity of sample material borne by the substrate.

According to a further aspect the present invention provides a computing device configured to carry out the method of the first aspect.

According to another aspect the present invention provides computer software for carrying out the method of the first aspect.

According to another aspect the present invention provides a computer program product comprising computer program code means to make a computer execute a procedure for estimating a quantity of sample material borne by an absorptive substrate, the computer program product comprising computer program code means for carrying out the method of the first aspect.

The present invention thus recognises that absorbent substrates, even high quality and highly certified filter papers currently available for applications referred to in the preceding, are not perfectly homogeneous materials. Consequently the absorptive capacity of the different filter papers can vary between manufacturers, and can vary between batches produced by the same manufacturer, and notably can even vary significantly across different sections of a single filter paper card.

In embodiments comprising a planar substrate such as filter paper, having first and second opposed surfaces, the first and second radiation are preferably each applied to the same surface of the filter paper, however other embodiments may provide for the first radiation to be applied to the first surface and the second radiation to be applied to the second surface. Similarly, the first radiation interaction measurement and the second radiation interaction measurement are preferably each obtained from the same side of the planar substrate, however other embodiments may provide for the first radiation interaction measurement and the second radiation interaction measurement to be obtained from opposing surfaces of the planar substrate. Still further embodiments provide for the first radiation interaction measurement and the second radiation interaction measurement to each be obtained from both the first surface and from the second surface of the planar substrate in order to measure both reflectivity and transmittivity, before and after the sample is applied.

The comparing of the first measurement and the second measurement may comprise using the first and second measurements in a formula to estimate the quantity of sample material borne by the substrate at the or each location. Estimating the quantity of sample material borne by the substrate may involve generating relative estimates, which compare the level of dried material contained in different target sites, wherein one site is estimated to have more or less sample material than another site, or is approximately the same. Alternatively estimating the quantity of sample material may involve generating an absolute value of sample material estimated to be borne by a particular portion of the substrate.

In some embodiments, comparing the first radiation interaction measurement with the second radiation interaction measurement to estimate the quantity of sample material borne by the substrate may also take into account the position of the target site relative to the centre or edge of the deposited sample, such as a bloodspot. Additionally or alternatively, comparing the first radiation interaction measurement with the second radiation interaction measurement may involve an assessment of a ratio of the first radiation interaction measurement to the second radiation interaction measurement. Additionally or alternatively, comparing the first radiation interaction measurement with the second radiation interaction measurement may involve an assessment of the absolute level of the first radiation interaction measurement and/or second radiation interaction measurement.

In some embodiments, the method of the present invention is additionally or alternatively applied to substrates used to bear a control material for a subsequent testing procedure. Such embodiments recognise that variable absorptivity of the substrate can affect control samples, thought to be by up to about 30% absorption variation, and that such measurements can improve reliability of control measurements thus improving test accuracy.

In embodiments of the invention, the first radiation and the second radiation may be substantially the same, or may differ in intensity, wavelength(s), duration or otherwise. The first and second radiation may in some embodiments be selected for the absorptive, transmissive or reflective interaction with the anisotropically distributed biological material and/or the anisotropic response of the substrate prior to sample absorption. For example, for selecting a sample of a blood specimen, the radiation may comprise a source of red light, wherein the interaction may be with the haem iron complexes of the material. Preferably the radiation is in the electromagnetic (EM) spectrum, although it is envisaged that other embodiments of the present invention may utilize other forms of radiation such as particle radiation. The EM radiation may in some embodiments be in the visible spectrum. However, in other embodiments UV/VIS, IR and/or high-energy (X- and y-) radiation may find utility. The radiation may be coherent radiation such as laser light or may have a significant dispersion. The radiation may be monochromatic or may comprise a spectrum of a selected band width or multiple spectral components.

One or both of the first and second radiation interaction measurements may comprise measurements obtained from the interaction of more than one type of radiation with the substrate. For example, a first scan may be performed using radiation in the red portion of the spectrum, and a second scan may be performed using radiation in the infrared portion of the spectrum, with the radiation interaction measurement comprising measurements obtained in response to both applied radiation types. Such embodiments recognise that different types of radiation may carry unique benefits in assessing the substrate and/or sample and that obtaining measurements of multiple radiation types may thus improve the ability to assess the absorptive capacity of the substrate before and after a sample is applied.

The radiation source may comprise any light emitting element such as a light emitting diode, laser or filament source. In the case of polychromatic sources the frequency or bandwidth may be provided by any suitable means such as a filter, grating or other monochromator. Alternatively the bandwidth or frequency reaching the detector may be selected by filter, grating or other monochromator after interaction with the sample. For example, for selection of samples for analysis from blood absorbed on filter paper the source may comprise a LED of median or notional wavelength of emission in the range of 600-800 nm, more preferably 700-750 nm. Such wavelengths have been observed to give a clearer, more distinct image of the blood captured with 3-dimensional aspect of the media.

The measurement of the interaction of the first radiation with the substrate, and of the interaction of the second radiation with the substrate and sample, may be by any suitable means determined by the choice of radiation and the nature of the interaction with the substrate and sample. For example, in the case of a blood specimen absorbed onto filter paper or the like, the measurement may be by means of a detector of the reflected and/or transmitted spectrum of an incident light source. Transmission through the specimen and substrate of light from the aforementioned LED may be detected by, for example a photodiode responding to a suitable wavelength. Available photodiodes for example may have a notional response of 700 nm, distributed between 400 nm to 870 nm.

The measuring of the interaction at a plurality of locations on the substrate may be achieved by any suitable means. For example, in some embodiments the substrate may be evenly illuminated and the plurality of locations scanned by a scanning detector of transmission or reflection as the case may be. The illumination may also be by a single point source which is scanned over the substrate in register with the scanning detector. Such scanning may be effected either by moving the source, or by moving the substrate. For reflective measurement the point source and the detector may be integrated in the same device component.

In alternative embodiments the measuring may comprise the use of an array of sources each element of which is associated with a discrete detector. Alternatively a first plurality of sources and a second plurality of detectors may be used, the first plurality not being equal to the second plurality, that is there may be greater or fewer detectors than sources. The array may comprise a two-dimensional array covering part or all of the field of interest. Alternatively the array may comprise a linear array adapted to be mechanically or optically scanned over the substrate, whether by array movement or substrate movement.

In yet further embodiments there may be provided a single source providing a pixelated incident radiation by means of a shadow mask or the like, and the detection being by means of multiple detectors in register with the shadow mask or by a scanning detector.

The radiation and the detection may be quantized to pixels of a size corresponding to the sample size to be punched out of the substrate for analysis. In this case the measure is of the average interaction between the radiation and the biological material over the sample area. Alternatively the radiation and detection may effect a quantization at smaller scales in order to give finer granularity and permit improved optimization of the sample selection.

The method of the present invention may be used to determine, for a chosen punch site, a predicted amount of sample which is present at that site and therefore a predicted amount of sample upon which a subsequent process such as an assay is operating. Alternatively, where it is known that the subsequent process requires a particular amount of sample, the method of the present invention may be used to search throughout the blood spot or sample site in order to find a punch site at which the desired amount of sample is predicted to be present, so that by punching at that site there is an improvement in the accuracy with which the desired amount of sample is delivered to the subsequent process.

The substrate may be analysed from one or both sides.

The present invention thus recognises that obtaining an understanding of the absorptive capacity of the filter paper, prior to sampling, at the site where the liquid biological sample is to be applied, can assist in improving accuracy of understanding of the amount of biological material contained in a filter paper disk removed from that site, or targeted for other sampling approaches such as direct absorption.

In some embodiments of the invention, the first radiation interaction measurement is obtained immediately prior to sample collection, to reduce the effects of environmental circumstances of card storage prior to use. Such embodiments recognise that a filter paper stored in moist environment will have a higher moisture content, and that when blood is applied the card will take longer to dry and the sample will spread further, reducing blood concentration in paper as compared to spotting of the paper when drier.

Alternatively, the first radiation interaction measurement may in some embodiments be obtained closer to the time of manufacture. Thus, according to a further aspect, the present invention provides a method for evaluating variations in absorptive capacity of an absorptive substrate, the method comprising:

prior to absorption of a sample by the substrate, directing a first radiation onto the substrate, the first radiation being configured to interact with the substrate to an extent relative to anisotropy of the substrate, and measuring interaction of the first radiation with the substrate at a plurality of measurement sites of the substrate to obtain a first radiation interaction measurement reflecting anisotropic absorptive capacity throughout the substrate.

This aspect of the invention recognises that filter paper or cards incorporating filter paper could be pre-screened at or soon after the time of manufacture, and only those filter paper/cards having anisotropic absorptive capacity which falls within predefined tolerances would be accepted or designated for certain uses requiring higher accuracy.

In embodiments where the card carries a RFID tag identifier, the first measurement may be stored within the RFID tag once obtained, until a later time at which the second radiation interaction measurement is obtained after the sample has been deposited upon the media.

### BRIEF DESCRIPTION OF THE DRAWINGS

An example of the invention will now be described with reference to the accompanying drawings, in which like reference numerals refer to like elements throughout the figures, and:

FIG. 1 is a partial side elevational view of an inspection apparatus of a first embodiment of the present invention;

FIG. 2 is a side elevational view of an inspection apparatus of the first embodiment incorporated into a further scanning head; and

FIG. 3 is a sectional top plan view of the scanning head of the apparatus of FIG. 2;

FIG. 4 is a flowchart illustrating a process in accordance with one embodiment of the invention; and

FIGS. 5a and 5b illustrate the average transmission number measured at each pixel location on a portion of a filter paper, for a prescan and postscan respectively.

### DESCRIPTION OF THE PREFERRED EMBODIMENTS

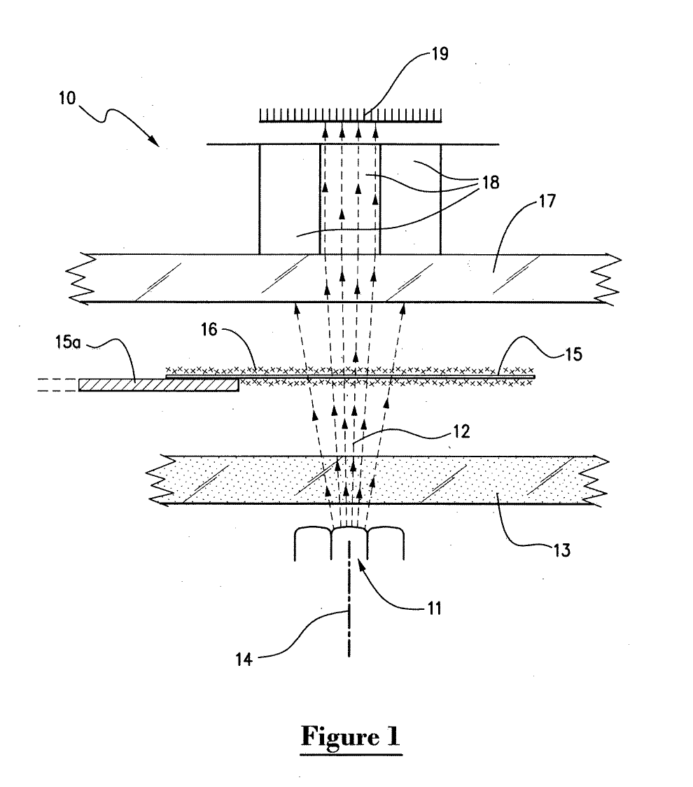

FIG. 1 illustrates an inspection apparatus 10 of a first embodiment which includes a light source 11 in the form of high intensity light emitting diodes (LEDs). The LEDs are suitably chosen such that the light 12 should not deflect by more than about 30° from the emitting axis 14 of the LEDs. The light 12 is passed through an opaque protective cover 13, such as an optical diffuser or frosted glass window or the like, which diffuses and improves uniformity of passing light and which protects the LEDs from dust and other adverse environmental conditions. At least one substrate 15 such as a paper filter card is placed in the path of the light 12. In a preferred arrangement, an array of approximately thirty (30) high intensity LEDs are mounted in a single row beneath the media 15, the media joined with a demographic form 15a along one edge which carries demographic details or similar information associated with the card. The media 15 when first measured in this way carries no biological material, and then is later spotted with a sample 16 of biological material for inspection, which is carried on and in the media 15 during a second measurement step.

The light passes through the media 15, and sample 16 if present, before again passing through an opposing protective glass window 17. Immediately adjacent to the opposing glass window 17 is a lens 18, being one of an array of similar lenses, which focuses the light that has passed through the media 15 parallel to the axis of the lens. The light passes through the lens 18 to an array of radiation detectors 19, whereby the light intensity is registered at a plurality of locations on a pixel-by-pixel basis.

Each pixel of the detector array 19, suitably of the photo-electric type, produces an analogue voltage proportional to the intensity/time of light exposure. The analogue voltage can be directed to an analogue-to-digital conversion circuitry to produce a grey scale image for further processing in a processor (not shown).

The lens 18 is arranged to produce a unit-magnification image from the surface of the filter card 15 to the detector 19. In this embodiment the image area is an array of approximately 2 mm diameter discs of light overlapping each other for the length of the lens 18. Each pixel suitably measures approximately 63.5×55.5 μm on 63.5 μm centre spacings, and the array is 1280×1 pixels in dimension. The total width of the scan will be around 81 mm in this embodiment; with the lens 18 being slightly wider than then detector array 19 to overcome edge effects.

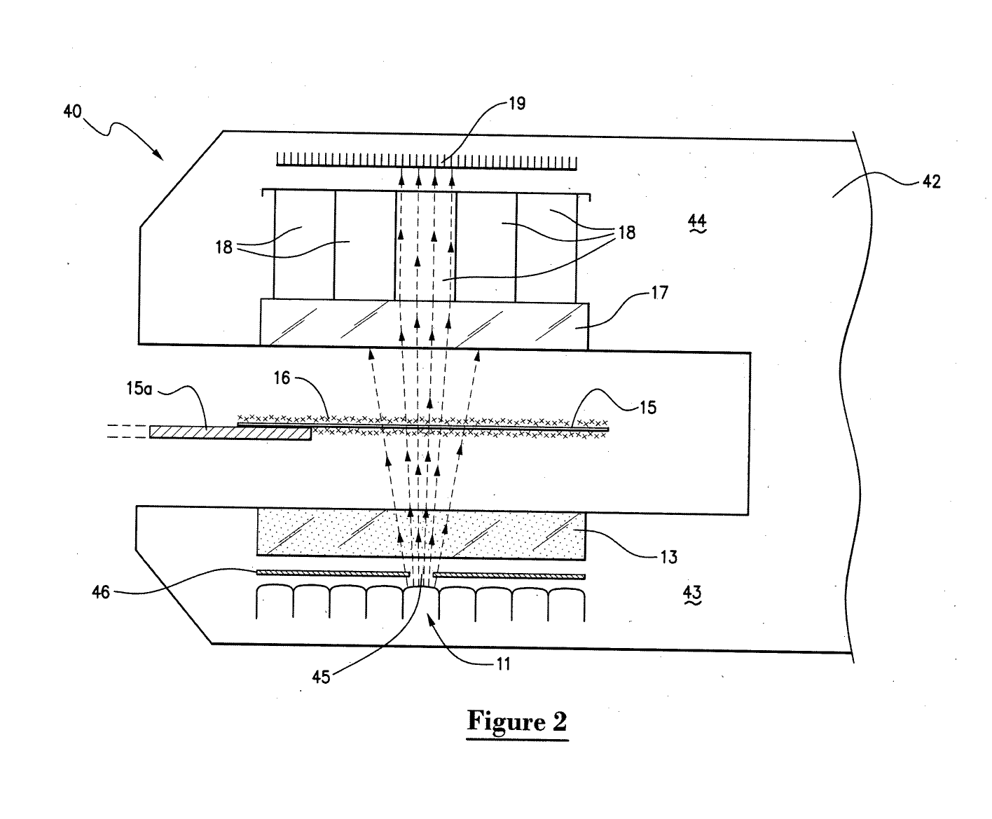

FIG. 2 illustrates an alternative embodiment wherein a scanning head 40 is provided with a support assembly 42 for the inspection apparatus 10 of the first embodiment. The support assembly is again in the form of a substantially C-shaped member or C-section 42 having opposing arms 43, 44. The array of LEDs, lenses, and detectors are disposed along the arms of the C-section as shown. The inspection apparatus 40 includes a light source 11, in the form of an array of LEDs, mounted on a first arm 43 of the support assembly 42. A light director 46, such as an LCD shutter, selectively provides a circular aperture 45 through which light is passed before impinging on the sample 16. The aperture 45 of the embodiment has a diameter of 3.2 mm which corresponds to the size of a disc shaped portion desired to be removed from the sample 16 for analysis. The sample 16 is carried on media, again in the form of a filter paper card 15 bearing a supporting edge piece 15a. The sample 16 may take the form of dried biological fluids, such as blood, urine or saliva.

The inspection apparatus 40 further includes a light detector 19 for measuring the intensity of light that is transmitted through the sample 16 and paper media 15. The measurement represents the average intensity of the light across the diameter of the circular aperture 45 through which light is passed. In the above example where the desired diameter of the disc shaped portion is 3.2 mm, then the diameter of the circular aperture through which light is passed is suitably also 3.2 mm, such that the resulting measurement is of the average intensity of light across the area of the aperture.

In the present embodiment the detector 19, formed by an array of photo-electric cells, is located on the reverse side of the media to which the light impinges and opposite to the aperture 45 and light source 11. The light source and director are fixed to a first arm 43 of the support member 40 and the detector 19 and associated array of lenses 18 is fixed to the second arm 44 in opposed relation to the source 11 and aperture 45.

It will be appreciated that the intensity of light measured after it passes through the media 15 where no sample is located is different to the intensity of light that has passed through media where sample 16 is located. The sensitivity of light measurement can be adjusted, so that differences in the amount of different samples can be recognized. In effect the measurement reflects the average amount of sample material across that circular area.

The C-shaped member 42 allows the edging 15a supporting the media 15 to pass between the light transmitting and sensing parts of the inspection apparatus 40. Either the media 15 can be passed through the C-section, which is fixed, or the C-section can pass over the media 15, as required. Under either approach, the light emitting part can make sufficient passes over the media to allow for a measurement of a plurality of portions on the media. Each pass is preferably at a speed selected to allow sufficient dwell time or integration time to permit accurate detection. Such measurements are preferably made at a sufficient number of positions upon the media 15, both before and after a sample is applied to the media, to permit targeting of different candidate portions of the sample.

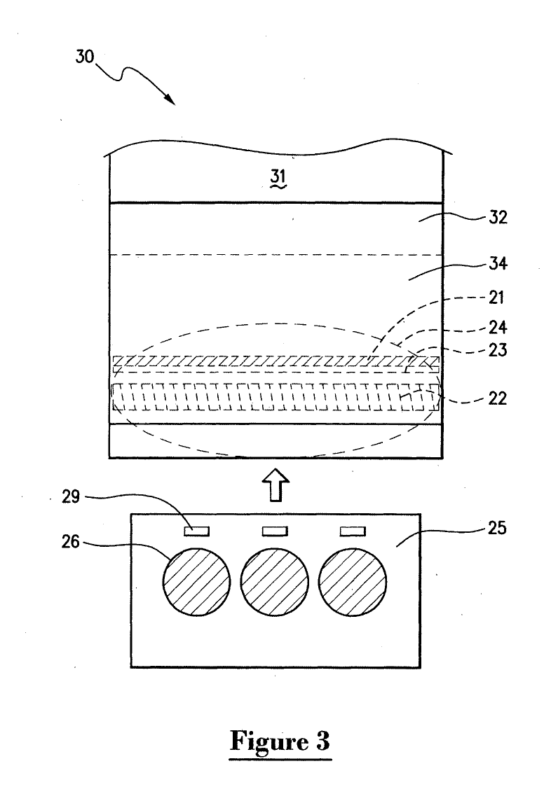

Referring now to FIG. 3, there is shown a plan view of a scanning head 30 with a support assembly 31 for the inspection apparatus 20 of the second embodiment, the scanning head using a reflective measurement approach. The support assembly is in the form of a substantially C-shaped member 32 having a lower arm (not shown) and opposing upper arm 34. The detectors 22, lens and associated glass window 24 are disposed in a free end of the upper arm 34 of the C-shaped member or C-section 32.

A medium in the form of a paper card 25 may be inserted either manually or automatically into the C-section 32 in a direction indicated by the arrow in FIG. 3. The card 25 includes designated areas 26 on the card to indicate the desired location for application of biological samples, such as blood. The number of designated areas 26 per card may vary, but preferably are in the range of 3-8, most preferably 4-6 inclusive. As the card 25 passes through the C-section and above the LEDs 11 of the primary light source, the LEDs pass light through the card and the samples 26, with that light being received through the lenses and by the detectors 22.

The secondary light source 21 can be used as an alternative source to facilitate detection of light reflected from the medium 25, as required. The sample card 25 can also include, in one embodiment, checkboxes 29 associated with each sample area 26. In the event that the laboratory has itself observed any inadequacy of a sample prior to scanning by the inspection apparatus 30, then the associated checkbox may be appropriately marked. The results of the scanning of the marked samples may then be discounted, so that no punching sites or sample portions are identified as being suitable from the whole sample area associated with a checked box.

In embodiments where the movement of the card 25 into the C-section 32 is automated, as the card is inserted between the opposed arms 33, 34 the scanner may be used to detect the leading edge of the card 25. The detected card edge is suitably utilised as a reference point for the identification of all other locations on the card 25. With automation, the card 25 can then be inserted predetermined standard distances into the identification apparatus 30, as required to ensure that scanning only occurs in the checkbox area (if checkboxes are incorporated in the sample card), and in other sample areas on the card where it is most likely that the sample is located, thus speeding the scanning process. The scanner set forth in FIGS. 1 to 3 is one of many different types of imaging devices which may be used for prescanning and postscanning in accordance with the present invention.

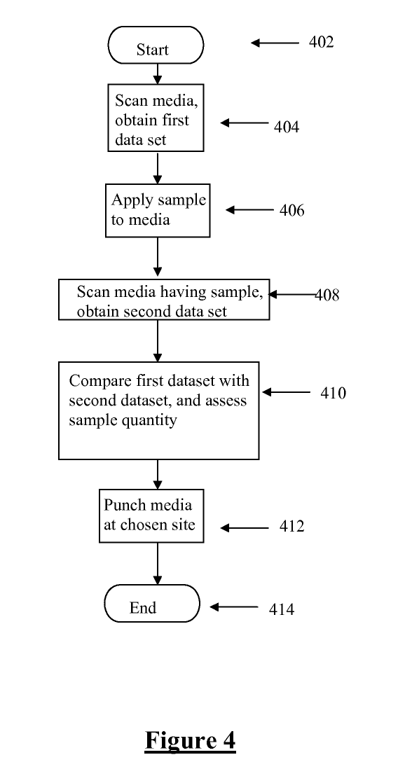

The present invention in one embodiment relates to a method for determining the amount of dried biological sample trapped in specific locations on a filter paper card, with improved accuracy than previously possible. As shown in FIG. 4, the method of this embodiment involves pre-screening the filter paper (404) to establish the absorptive capacity at all locations of interest on the card. After the sample is applied (406) a postscan screening of all possible sampling locations within the biological spot is obtained (408). The prescan (first) data set is compared at 410 with the postscan data set to assess the quantity of sample material present at each of the scanned locations.

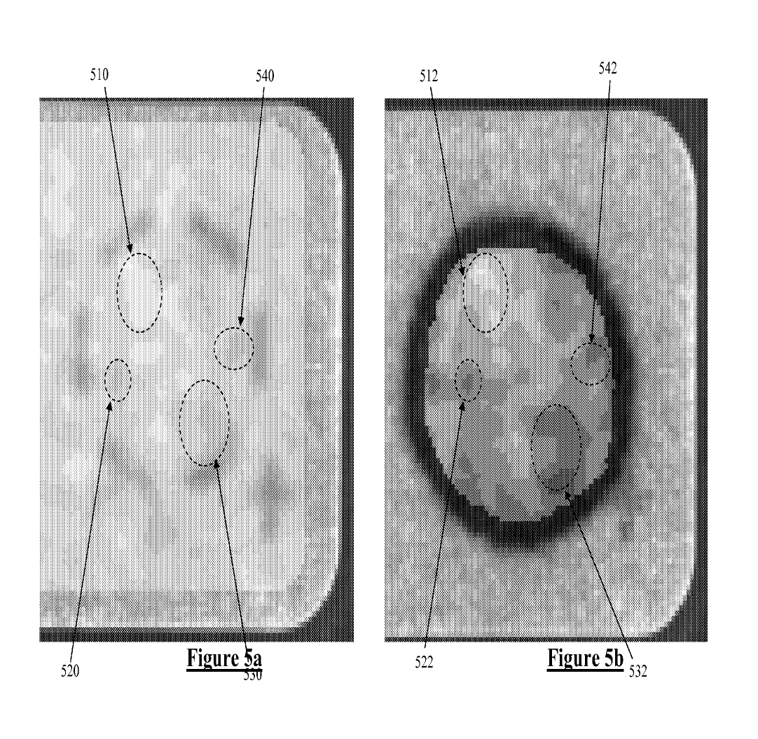

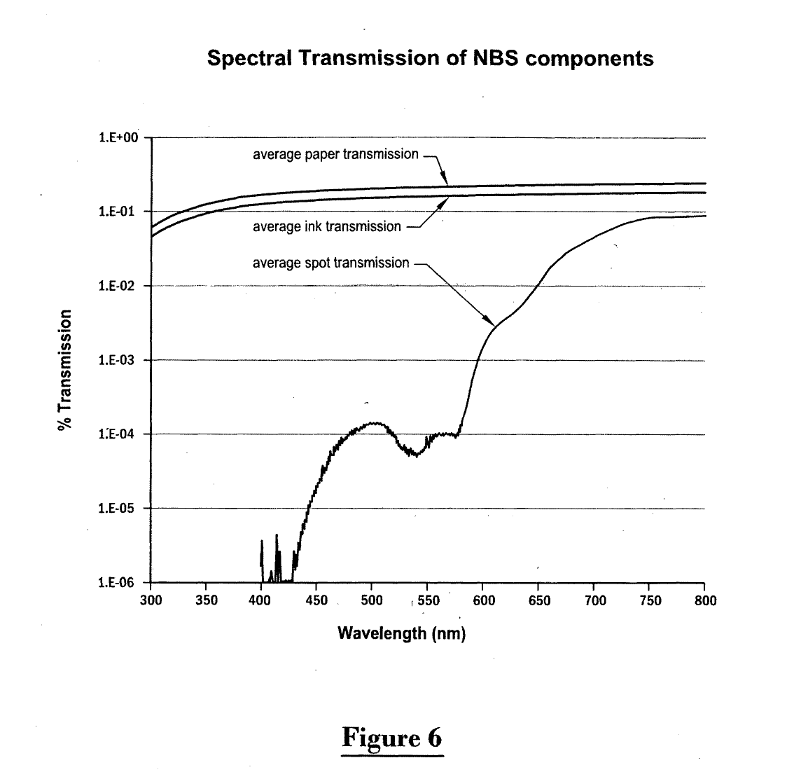

FIGS. 5a and 5b illustrate the average transmission number measured at each pixel location on a portion of a filter paper, for a prescan and postscan respectively. Lighter portions indicate a higher transmission number, as returned by areas having higher measured transmittivity. Darker portions reflect lower transmittivity. As can be seen in FIG. 5a, prior to any sample being applied, the filter paper has a certain amount of variation in transmittivity at different locations in the filter paper. When comparing the prescan ATN map of FIG. 5a to the postscan ATN map of FIG. 5b, it is noted that there is some correlation between transmittivity at common locations on the paper. For example, similarity exists between the region indicated by 510 in the prescan and the region indicated by 512 in the postscan. Further, similarities exist between the region indicated by 520 in the prescan and the region indicated by 522 in the postscan, between the region indicated by 530 in the prescan and the region indicated by 532 in the postscan, and between the region indicated by 540 in the prescan and the region indicated by 542 in the postscan, respectively. This illustrates that past approaches, which consider only the postscan ATN, fail to differentiate between the transmittivity of the applied sample and the transmittivity of the media itself.

The punch site may be chosen by reference to these results. For example if a subsequent assay process requires a predefined amount of sample, then the predicted sample quantity may be searched throughout the entire sample location in order to identify a punch site within which the predefined amount of sample is predicted to be present based on the scan data, or as close to that predefined amount as possible. The punch site determined in this way may be used to control a punch integrated with the scanner into a single device, or may be used to control a punch separate to the scanner, or may be presented to a human operator of a manual punch.

Alternatively, the punch site may be selected by an alternative method and the scan data obtained by the present invention may be used to estimate an amount of sample present at that site, to assist accurate interpretation of results obtained from a subsequent assay or other process performed on the punched disc.

In one embodiment of this method, filter paper cards are screened individually before any sample is applied, the absorptive capacity measurements of each card are recorded, and the card is identified by way of a specific and unique identifier such as a barcode or RFID identifier.

In another embodiment, filter paper or cards incorporating filter paper are pre-screened at time of manufacture, and only filter paper/cards that fall within predefined laboratory-accepted tolerances with respect to variations in absorptive capacity are accepted and correspondingly labelled for those uses requiring higher accuracy. Another option is to screen the whole roll of filter paper across its full width while still in bulk, with only bulk rolls of paper which meet tolerances then being selected to be made into cards.

Embodiments of the present invention may thus provide some ability to determine the amount of blood in an area of filter paper, based on a comparison of a “prescan” of the unspotted card with a scan of the spotted card.

Experiments to date confirm the ability to identify, with improved accuracy, variations in the density of the filter paper across different areas of the unspotted cards. A plurality of cards have been tested, by pre-scanning the cards to determine those areas displaying different density, spotting the cards in the variable density areas with a controlled amount of blood of the same hematocrit, drying the cards in the same environment, and scanning afterwards (referred to as a postscan) and recording the light transmission in those areas. Punch sites were then identified from the prescan and postscan data, with disks punched from those sites and testing performed of blood-related compounds captured in the disks as a measure of actual sample content within the disk.

Prescanning results reveal that variations in the transmission of light through commercially available filter paper are evident, often on a pixel by pixel basis. At the time of pre-screening the identity of the card is fixed by using a barcode or RFID tag, and the pixel by pixel transmission information is recorded as the first radiation interaction measurement. The first radiation interaction measurement is then compared with a second radiation interaction measurement which is obtained after sampling, and the differences compared with configurable light transmission threshold levels. The card identity for each measurement is confirmed by the barcode/RFID tag.

The prescan measurements of a large number of blank filter paper cards can be efficiently performed in a bulk manner at a central location. In such embodiments, it is important to ensure that the instrument at the central location used to perform the prescan and the instrument used to perform the postscan are carefully calibrated, to ensure the respective measurements can validly be compared.

From an analysis of light transmitted through dried blood spots, and detected and measured on the other side, it has been observed that significant variations can appear to exist in the measurements within a blood spot, even when adjacent parts of the blood spot appear to be equivalent to the naked eye. This variation may be attributed to a variation in the absorptive characteristics of different parts of the same filter paper.

There have been previous reports prepared by the US Centre for Disease Control comparing and contrasting the absorption levels of alternative commercially available filter papers, including those offered by Whatman GE and Ahlstrom. Specifications provided by these suppliers for filter papers used for purposes such as neonatal screening suggests a variation in uptake of blood materials of up to 30%. It had traditionally been assumed that this variation might occur between batches of paper, or lots. In fact, the present inventor's use of transmitted light has suggested that significant variations can occur within and across small areas of filter paper on the same filter paper card. On first analysis, variations in light transmission measurement can be of this magnitude, in immediately adjacent areas of filter paper.

Areas of filter paper which allow less light to be transmitted may do so because the filter paper itself is more dense, because of the nature of the matrix construction at that point, or because the paper is thicker at that site. Filter paper that is more dense might tend to absorb and trap less blood material as the latter dried, and filter paper where the matrix does not allow for the passing of light would not as easily allow for the encroachment of liquid blood during spotting. In contrast, areas of filter paper that were thicker than others are likely to transmit less light, but would potentially absorb more sample.

In order to test these theories in more detail, a number of simple experiments were undertaken. Firstly, in order to test the theory that unspotted target areas with significantly lower transmission areas were more dense than other areas, examples of such target areas were identified which corresponded to the size of a typically available filter paper punch, and punched out using a standard diameter filter paper punch available from BSD Robotics. The punched disks were then weighed to confirm the mass of the filter paper in each punched disk. This was undertaken on the basis that while the punching of the disks would lead to compression, it would not lead to any change in the mass of the filter paper material.

Weighing of the punched disks was undertaken to milligram level, and while measurable variations were found, there appeared to be no clear connection between the light transmission levels, and the weight of the disk punched from the target areas. It was concluded that the construction of the matrix at those points was a more likely source of the variation in light transmission levels than a variation in density.

Specifications for commercially available filter paper do not nominate the thickness tolerance, but experience in the manufacture of instruments designed to handle this paper prior to punching suggests that this variation can be as great as 10%. To initially test the theory of varying thickness, scans were undertaken of a number of individual pieces of filter paper at specific target areas on the cards. These pieces of filter paper were then progressively brought together between the scanner arms in such a way that the target areas were in line vertically between the scanner arms. With the addition of each piece of additional paper, the target areas were scanned. As a result of this process, the relationship between paper thickness and light transmission could be better understood. The conclusion from this initial test was that light transmission is related to paper thickness in such a way that at the typical thickness (0.4-0.5 mm) and light transmission of filter paper, a 100% increase in paper thickness resulted in a 30% loss in light transmission. A 10% variation in thickness would likely result in a small variation in light transmission, e.g. up to 5%. This was noted as a possibility to be taken into account, when the results of testing of blood sample volumes were being considered.

An additional variation that could potentially affect the tests was also acknowledged in the case of scanning of unspotted filter paper. It is known that blank paper can act as a diffuser of light, i.e. light is refracted and diffused in various directions when being transmitted through filter paper. It is therefore possible that measurement of light transmission through unspotted filter paper could lead to misleading results if it incorporated light transmitted through other sections of the paper, rather than the targeted areas to which the measurement was being attributed. On the other hand, if the refraction/diffusion of light through unspotted filter paper was consistent i.e. uniform across filter papers, then this effect would be able to be accounted for, both in any comparison of target sites, and in absolute terms. In order to test this question, a special test was performed whereby a single LED (from within the LED array normally illuminated in the BSD instrument) was allowed to transmit light through blank and spotted filter paper as the scanner arms traveled over the filter paper card. At each pixel point on the travel, the measurements of transmitted light, as recorded at each of the detectors in a line perpendicular to the line of travel, was mapped. Testing indicates that this diffusion occurs in a very uniform manner for specific filter paper types. This effect in diffusing the radiation as it passes occurs significantly with blank paper, but to a much lower degree with filter paper involving dried sample. The uniformity of this effect gives confidence that using unadjusted pixel-by-pixel results gives an accurate portrayal of the extent of variation between pixels. The most significant effect of the diffusion however occurs at the edge of the blood spot where diffusion from blank paper surrounding the sample somewhat distorts the scan results for about 1.25 mm of pixels into the spot, reaffirming the practise that these areas at the edge of the spot should not be included in any calculation for the purposes of determining the amount of blood material in a possible disk to be punched.

In the preparation of the apparatus and methodology used to test the above theories, at least one other important observation was made in relation to the system used for filter paper scanning That observation relates to the importance of maintaining the filter paper card as close to horizontal as possible during each scanning operation. With framed filter paper cards, the effect of the frame is to hold the filter paper essentially in a single plane. However for unframed cards when dried bio-sample, and especially blood, is applied to unframed cards those cards tend to warp. For the purposes of the current analysis, all testing was performed with framed cards, with a view to eliminating any possibility that there may be any warp factor affecting the light transmission outcomes.

The test used to identify the extent of paper-related absorption variation involved the following steps:
a) Scanning a selection of blank cards using a near infrared light source at 730 nm, and recording the amount of light transmitted through every pixel within the target areas on the cards;
b) Using a method that allows for light transmission measurements to be aggregated for an area reflecting a typical punch size (e.g. a circle of 3.2 mm diameter) and then creating an average transmission number (ATN) for all possible punch positions on the card (including punch positions spaced apart by less than 3.2 mm);
c) Reviewing the ATN for all possible punch sites, and identifying a selection of non-overlapping punch sites which displayed either very similar ATNs, or significantly different ATNs (e.g. an ATN variation of 10% or more);
d) Arranging for the cards to be spotted at the same time, using a blood with a known hematocrit, and incorporating a number of known markers that could later be detected with precision using tandem mass spectrometry (TMS). Phenylanaline was selected as being a suitable marker due to being strongly indicative of blood volume and being known to spread evenly within the blood sample;
e) Ensuring that the spotting was performed by the same lab staff member, with each spot being accurately dispensed by pipette, using the same technique;
f) Allowing the cards to air dry together in normal lab conditions for 2+ hours;
g) Rescanning the cards after spotting, and reviewing the spotted ATNs at the punch sites selected in step (c) above,
h) Confirming the existence on some cards of sites where the spotted ATN if considered alone might suggest that there was more blood at one site than another, (i.e. the spotted ATN at Site A was lower than at Site B), but where the differences between the unspotted ATN and spotted ATN at Site B was greater, (because the unspotted ATN at Site B was significantly higher than at site A) suggesting that there had been more material applied to Site B;
i) Arranging for selected target sites to be punched from all cards, using a commercially available punch with light targeting system, and for these disks to be punched into a standard 96 well plate suitable for use in TMS equipment.
j) Ensuring that the targeted and punched sites included a selection of sites from the centre of the blood spot and also from areas between the centre of the blood spot and the edge of the blood spot;
k) Rescanning the punched cards to establish the differences between unspotted ATNs and spotted ATNs at the actual punched locations (given that these might have varied even slightly from the selected sites);
l) Analysing the dried blood captured on each of the disks punched from the cards using TMS processes, and on the basis of the extent of the markers found; and
m) Assessing the correlation between the results from the disks processed in line with k) above and the ATN information as determined in k) above.

In relation to (j) above, advice from laboratories indicated that while virtually all had a policy not to punch disks adjacent to the edge of a dried blood spot, many had a policy to avoid punching from the centre of dried blood spots. It was intended that the results of the process described above would test theories in relation to the usefulness of the average transmission numbers (ATNs) at a target site before and after spotting, in forecasting the volume of blood that had been captured at that target site.

The process was repeated for two brands of commercially available filter paper promoted for purposes such as neonatal screening, these being:

Ahlstrom 226

Munktell TFN

In tables 1 to 7 below the letters above the test results (A/A, A/B, B/A etc) relate to the orientation of the card during application of blood and scanning One side of the card usually has printed circles to help guide the collectors as to where to place the blood. That side we have referred to as side A. A notation of A/B indicates that the card was spotted onto side A and that the scanning (both before and after the blood was applied) was done from side B, i.e. the opposite side. After the first round of tests (Tables 1 to 2), where all were approached from an A/A basis, we subsequently tested cards by scanning from both sides.

Testing involved the use of control blood to create bloodspots on two paper types, namely Ahlstrom 226, and Munktell TFN. For most of the cases, 50 μl of control blood was applied. In the case of the first 91 Ahlstrom samples, samples of 20 μl were applied, and so results for this test appear separately. Disks were punched from blood spots created on each type of card.

Results.

Ahlstrom 226.

Initial results were generated for 91 samples from framed cards manufactured in March 2010 and tested in August-October 2011. Before spotting took place, up to four punch sites had been identified on each of 24 cards. Of the total of 94 samples actually punched, the laboratory rejected 3 results as invalid, due to analytical error. Prior to spotting, ATNs had been generated for each of the 91 valid target sites, and for blank cards, these ranged from 2306 (the darkest target site) to 2867 (the lightest). After spotting with 20 μl of standard hematocrit blood, the cards were again scanned and the ATNs at the target sites ranged from 565 (the darkest spot) to 925.

The marker that was selected for use as an accurate measure of blood volume, based on its extensive use for that purpose in newborn screening and some other applications, was phenylalanine (PHE). Marker values across the 91 valid samples ranged from 192 to 259, with a mean of 222, a Standard Deviation of 13.6 and CV of 6.1%, as shown in Table 1.

TABLE 1

| | Ahlstrom Test 1-A/A. N = 91 | | |
|---|---|---|---|
| | Lab | BloodScan | w PreScan |
| Average PHE | 222.51 | 222.51 | 222.51 |
| SD | 13.64 | 10.20 | 8.95 |
| CV % | 6.13 | 4.58 | 4.02 |

Multiple regression analysis was used to analyse the strength of the relationship between a number of variables including:
1) initial (blank) filter paper ATN level
2) the after-spotting blooded ATN value
3) the reduction in ATN value as a result of spotting (either reflected in the ATN difference, or as a ratio of spotted to unspotted ATNs).
4) The distance between the centre of the blood spot and the centre of the target disk.

The detailed test results can be summarised as follows:
a) There was a relationship between the ATN of all blooded disks (after spotting) and the PHE values, with adjusted R² value of 0.44. When all disks that had their centre within 1 mm of the centre of the blood spot were eliminated from the analysis, the result was a strong improvement in the correlation with an adjusted R² value of 0.89.
b) There was a clear relationship between the (blank) filter paper ATN level and the extent to which the ATN for the same target site would reduce when a given amount of blood was captured at the site. At higher initial ATN levels, the reduction in ATN levels would be greater, given the same level of blood content.
c) There was generally an identifiable relationship between the initial ATN, and the reduction in ATN value after spotting (either reflected in the ATN difference, or as a ratio of spotted to unspotted ATNs) and the resultant marker values, with R² values of 0.56.

When all disks that had their centre within 1 mm of the blood spot were eliminated from the analysis, there was a very strong relationship found between the resultant marker values and a combination of multiple variables including a) the initial ATN, b) the reduction in ATN value after spotting (either reflected in the ATN difference, or as a ratio of spotted to unspotted ATNs) and c) the distance from the disk centre to the blood spot centre, with R² values of 0.94.

Munktell TFN.

Initial results were generated for 93 samples from framed cards with manufacturer's validity till December 2013. Before spotting took place, up to four punch sites had been identified on each of 25 cards. Of the total of 94 samples actually punched, one disk straddled the edge of the blood disk and was eliminated. Prior to spotting, ATNs had been generated for each of the 93 valid target sites, and for blank cards, these ranged from 2211 (the darkest target site) to 3013 (the lightest). After spotting with 50 μl of standard hematocrit blood, the cards were again scanned and the ATNs at the target sites ranged from 517 (the darkest spot) to 1262.

PHE values across the 93 valid samples ranged from 184 to 272, with a mean of 233, a Standard Deviation of 18.28 and CV of 7.9% as shown in Table 2.

TABLE 2

| | Munktell Test 1-A/A. N = 93 | | |
|---|---|---|---|
| | Lab | BloodScan | w PreScan |
| Average PHE | 232.74 | 233.02 | 233.02 |
| SD | 18.28 | 7.08 | 6.93 |
| CV % | 7.86 | 3.04 | 2.97 |

The detailed test results of the multiple regression analysis were very similar to those obtained with the Ahlstrom paper, and can be summarised as follows:
a) There was a strong relationship between the ATN of all blooded disks (after spotting) and the PHE values, with adjusted R² value of 0.84. When all disks that had their centre within 1 mm of the centre of the blood spot were eliminated from the analysis, the result was a slight reduction in the correlation, with an adjusted R² value of 0.83.
b) There was a clear relationship between the (blank) filter paper ATN level and the extent to which the ATN for the same target site would reduce when a given amount of blood was captured at the site. At higher initial ATN levels, the reduction in ATN levels would be greater, given the same level of blood content.
c) There was generally an identifiable relationship between the initial ATN, and the reduction in ATN value after spotting (either reflected in the ATN difference, or as a ratio of spotted to unspotted ATNs) and the resultant marker values, with R² values of 0.85.

When all disks that had their centre within 1 mm of the blood spot were eliminated from the analysis, there was a very strong relationship found between the resultant marker values and a combination of multiple variables including a) the initial ATN, b) the reduction in ATN value after spotting (either reflected in the ATN difference, or as a ratio of spotted to unspotted ATNs) and c) the distance from the disk centre to the blood spot centre, with R² values of 0.84.

Further tests were conducted with the same types of paper.

TABLE 3

| | Ahlstrom Test 2-B/A. N = 76 | | |
|---|---|---|---|
| | Lab | | |
| | 120811-1 | BloodScan | w PreScan |
| Average PHE | 289.66 | 289.66 | 289.66 |
| SD | 12.60 | 10.07 | 8.98 |
| CV % | 4.35 | 3.48 | 3.10 |

TABLE 4

| | Munktell Test 2-A/B. N = 78 | | |
|---|---|---|---|
| | Lab | | |
| | 120811-2 | BloodScan | w PreScan |
| Average PHE | 249.51 | 249.51 | 249.51 |
| SD | 21.37 | 10.71 | 10.29 |
| CV % | 8.56 | 4.29 | 4.12 |

Consolidated results for over 300 samples on these types of papers are set out in Tables 5-6.

TABLE 5
Ahlstrom 226. N = 167

| | | | Based on |
|---|---|---|---|
| | | Based on | Blood Scan and |
| Measure | Control Blood | Blood Scan | Blank Card Scan. |
| Average | 253.1 | 253.1 | 253.1 |
| Standard Deviation | 14.80 | 12.60 | 10.28 |
| CV | 5.85 | 4.98 | 4.06 |
| R Square | | 0.88 | 0.92 |

TABLE 6
Munktell TFN. N = 171

| | | | Based on |
|---|---|---|---|
| | | Based on | Blood Scan and |
| Measure | Control Blood | Blood Scan | Blank Card Scan. |
| Average | 240.5 | 240.5 | 240.5 |
| Standard Deviation | 18.4 | 9.56 | 8.95 |
| CV | 7.65 | 3.98 | 3.72 |
| R Square | | 0.80 | 0.83 |

From Tables 5-6 it is observed that significant variations were found to exist in the actual level of PHE (measured in micromoles) found in 3.2 mm disks punched from control blood material carrying PHE. The amount of PHE per unit of blood was controlled, so that if the paper was totally uniform the measured amounts of PHE should have been the same and not possessed such variations.

A regression analysis was performed involving the actual measured PHE values and the blood scan result, to create a formula permitting prediction of the expected variations based on the prescan and postscan results. Notably, the fourth column of Tables 5-6 show the additional benefit obtained by conducting the prescan in addition to the postscan.

The present method and the above results thus show that the possibility to normalise the results to account for variations in the absorptive capacity of the filter paper can permit improved accuracy in the assessment of results, particularly in quantitative assay methods and the like. For example, the prescan technique is able to be used to predict with improved accuracy the amount of dried blood material contained in punched disks. These embodiments thus recognise that there is value in being able to place a more consistent amount of dried blood material into laboratory assays.

The scanner and punch may include software permitting an operator to specify a target level of dried blood material, on an assay-by-assay basis. The BSD 300PLUS represents an instrument which may be configured with such capability.

It is noted that in the described embodiments, within a 3.2 mm diameter disk this technique considers almost 200 pixels, and that across that 3.2 mm diameter disk only an average of all the localised variations is applicable. However, with the trend towards smaller punch disks, the occasionally significant pixel-to-pixel variations will be more influential and the present invention may thus be of even greater utility when applied with smaller punched disks.

It will be appreciated by persons skilled in the art that numerous variations and/or modifications may be made to the invention as shown in the specific embodiments without departing from the spirit or scope of the invention as broadly described. For example, in the case of buccal samples a fluorescing reflective imaging approach may be apt, as dried saliva for example may not be visible on clear paper but when excited by red/green light imaging of a fluorescing response may be applied.

Moreover, alternative embodiments may be implemented with alternative light source characteristics. FIG. 6 illustrates the spectral transmission of light in the 300-800 nm range through paper, ink, and red blood spots from new born screening (NBS). As can be seen, the absolute transmission of light through a spotted sample card depends not only on transmitted light intensity and period of exposure, but also on the frequency of light selected. As the capability of the system to differentiate between varying blood samples is reduced with reducing absolute light transmission, embodiments using wavelengths other than the 730 nm discussed in the preceding preferably take into account the spectral variation in transmittivity by adjusting factors such as the source intensity and/or exposure time accordingly. The ability of such alternative embodiments to use these other factors to compensate for lesser wavelengths will however depend on other issues including (a) whether increased intensity will generate unreadable transmission levels through the surrounding blank filter paper by saturating the receivers, (b) whether there is bleeding of high intensity surrounding light into the very edges of the blood spot, and/or (c) the timeframe available to read transmission levels.

The present embodiments are, therefore, to be considered in all respects as illustrative and not restrictive.

1. A method for estimating a quantity of sample material borne by an absorptive substrate, the method comprising:
prior to absorption of a sample by the substrate, directing a first radiation onto the substrate, the first radiation being configured to interact with the substrate to an extent relative to anisotropy of the substrate, and measuring interaction of the first radiation with the substrate at a plurality of measurement sites of the substrate to obtain a first radiation interaction measurement;
applying a sample to the substrate to cause absorption of sample material into the substrate at at least one of the measurement sites;
after absorption of the sample by the substrate, directing a second radiation onto the substrate, the second radiation being configured to interact with the substrate and to interact with the absorbed sample borne by the substrate to an extent relative to anisotropy of the substrate and the absorbed sample, and measuring interaction of the radiation with the substrate and sample at the plurality of measurement sites of the substrate to obtain a second radiation interaction measurement; and
comparing the first radiation interaction measurement with the second radiation interaction measurement to estimate a quantity of sample material borne by the substrate.

2. The method of claim 1, wherein the first radiation and second radiation are each applied to a first surface of the substrate.

3. The method of claim 1, wherein the first radiation is applied to a first surface of the substrate and the second radiation is applied to a second surface positioned opposite the first surface.

4. The method of claim 2 wherein the first radiation interaction measurement and the second radiation interaction measurement are each obtained from a second side of the planar substrate opposite the first surface, so as to measure transmitted radiation.

5. The method of claim 2 wherein the first radiation interaction measurement and the second radiation interaction measurement are each obtained from a first side of the planar substrate so as to measure reflected radiation.

6. The method of claim 1 wherein the first radiation interaction measurement and the second radiation interaction measurement are each obtained from both sides of the planar substrate in order to measure both reflectivity and transmittivity, before and after the sample is applied.

7. The method of claim 1 wherein the comparing of the first measurement and the second measurement comprises using the first and second measurements in a formula to estimate the quantity of sample material borne by the substrate at the or each location.

8. The method of claim 1 wherein the comparing of the first radiation interaction measurement with the second radiation interaction measurement comprises an assessment of a ratio of the first radiation interaction measurement to the second radiation interaction measurement.

9. The method of claim 1 wherein the comparing of the first radiation interaction measurement with the second radiation interaction measurement comprises an assessment of the absolute level of the first radiation interaction measurement and/or second radiation interaction measurement.

10. The method of claim 1 wherein the comparing of the first radiation interaction measurement with the second radiation interaction measurement takes into account the position of the target site relative to the centre or edge of the deposited sample.

11. The method of claim 1 wherein the sample material is a control material deposited on the substrate for a subsequent testing procedure.

12. The method of claim 1 wherein the first radiation and the second radiation are of substantially the same intensity, wavelength and duration.

13. The method of claim 12 wherein the first radiation and the second radiation are of a wavelength, or comprise a band having a band centre, which is in the range of 650-800 nm.

14. The method of claim 13 wherein the first radiation and the second radiation are of a wavelength, or comprise a band having a band centre, which is in the range of 700-750 nm.

15. The method of claim 14 wherein the first radiation and the second radiation are of a wavelength, or comprise a band having a band centre, which is in the range of 725-735 nm.

16. The method of claim 1 wherein at least one of the first radiation interaction measurement and the second radiation interaction measurement comprises measurements obtained from the interaction of more than one type of radiation with the substrate.

17. The method of claim 1 further comprising determining, for a chosen punch site, a predicted amount of sample which is present at that site and therefore a predicted amount of sample delivered to a post-punching process.

18. The method of claim 1 further comprising searching throughout the sample to find a punch site at which a desired amount of sample is predicted to be present, and punching at that site.

19. The method of claim 1 wherein the first radiation interaction measurement is obtained immediately prior to sample collection.

20. A method for evaluating variations in absorptive capacity of an absorptive substrate, the method comprising:
prior to absorption of a sample by the substrate, directing a first radiation onto the substrate, the first radiation being configured to interact with the substrate to an extent relative to anisotropy of the substrate, and measuring interaction of the first radiation with the substrate at a plurality of measurement sites of the substrate to obtain a first radiation interaction measurement reflecting anisotropic absorptive capacity throughout the substrate.

21. The method of claim 20 when used to pre-screen substrates at or soon after a time of manufacture, and further wherein only those substrates having anisotropic absorptive capacity which falls within predefined tolerances are accepted or designated for certain uses requiring higher accuracy.

22. The method of claim 1, further comprising storing the first measurement within a RED tag identifier once obtained, until a later time at which the second radiation interaction measurement is obtained after the sample has been deposited upon the media.

23. A substrate scanning device configured to carry out the method of claim 1.
